SEQUENCE LISTING (11 sequences)

SEQ ID NO: 1 - protein, 285 aa, Streptomyces griseus
MAPDIPLANVKAHLTQLSTIAANNGGNRAHGRPGYKASVDYVKAKLDAAGYTTTLQQFTSGGATGYNLIADWPGGDPNKVLMAGAHLDSVSSGAGINDNGSGSAAVLETALAVSRAGYQPDKHLRFAWWGAEELGLIGSKYYVNNLPSADRSKLAGYLNFDMIGSPNPGYFVYDDDPVIEKTFKDYFAGLNVPTEIETEGDGRSDHAPFKNVGVPVGGLFTGAGYTKSAAQAQKWGGTAGQAFDRCYHSSCDSLSNINDTALDRNSDAAAHAIWTLSSGTGEPPT

SEQ ID NO: 2 - protein, 299 aa, Aeromonas proteolytica
MPPITQQATVTAWLPQVDASQITGTISSLESFTNRFYTTTSGAQASDWIASEWQALSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQ

SEQ ID NO: 3 - protein, 317 aa, Serratia marcescens
MEQLRGLYPPLAAYDSGWLDTGDGHRIYWELSGNPNGKPAVFIHGGPGGGISPHHRQLFDPERYKVLLFDQRGCGRSRPHASLDNNTTWHLVADIERLREMAGVEQWLVFGGSWGSTLALAYAQTHPERVSEMVLRGIFTLRKQRLHWYYQDGASRFFPEKWERVLSILSDDERKDVIAAYRQRLTSADPQVQLEAAKLWSVWEGETVTLLPSRESASFGEDDFALAFARIENHYFTHLGFLESDDQLLRNVPLIRHIPAVIVHGRYDMACQVQNAWDLAKAWPEAELHIVEGAGHSYDEPGILHQLMIATDRFAGK

SEQ ID NO: 4 - protein, 295 aa, Pyrococcus furiosus
MDTEKLMKAGEIAKKVREKAIKLARPGMLLLELAESIEKMIMELGGKPAFPVNLSINEIAAHYTPYKGDTTVLKEGDYLKIDVGVHIDGFIADTAVTVRVGMEEDELMEAAKEALNAAISVARAGVEIKELGKAIENEIRKRGFKPIVNLSGHKIERYKLHAGISIPNIYRPHDNYVLKEGDVFAIEPFATIGAGQVIEVPPTLIYMYVRDVPVRVAQARFLLAKIKREYGTLPFAYRWLQNDMPEGQLKLALKTLEKAGAIYGYPVLKEIRNGIVAQFEHTIIVEKDSVIVTTE

SEQ ID NO: 5 - protein, 793 aa, Lactobacillus helveticus
MKYNQYAYVETDFQQQVKELIDINFLPKNYQVWDFSSLLAKLVKNAIAEAKTDAAKNAKLAEFAVSDHQTLADFLKEKPTEIGTKQFYNVALQLLGYHVHYDYDFADPTGFMQRNALPFLQDISDNQKLISAFYRLLNTRAKNGQILLDVMAGKGYFTQFWGQNKFKFFNGKSIPVFDTNKVIREVVYVETDLDTDHDGKSDLIQVTVFRPEETNKGLKVPALYTASPYFGGIIANEKRNHNVDENLSDSTEWNDPQYVHSPIVKAEKPDGSSRPATEEAVHKSSYPLNEYMLARGFASVFAGAIGTRGSDGVRITGAPEETESAAAVIEWLHGDRVAYTDRTRTVQTTADWCNGNIGMTGRSYLGTLQIAIATTGVKGLKTVVSEAAISSWYDYYREHGLVIAPEACQGEDLDLLAETCQSNLWDAGSYLKIKPEYDKMQKQLREKEDRNTGQYSDFWEARNYRHHADGIKCSWISVHGLNDWNVKPKNVYKIWQLVKKMPMKHHLFLHQGPHYNMNNLVSIDFTDLMNLWFVHELLGIENNAYNQWPTVMIQDNLQADKWHEEPDWSNDLGQEKIYYPTDEGELFQDGNGKAQKSFTDVGGIEFKKAGISESDWQYKFICGDEKWAKPSLRFETDEFTHPTTIVGRPEVKVRVSASLPKGEISVALVELGERQRLTATPKFLMHGGQELGYRFGTDTLQEFVPDKKTKAKLITKAHMNLQNFKDMKKPEAIDADKFYDLDFLLQPTYYTIPSGSKLALIIYSTDQGMTKRPLEDETYTIDLANTEIKFYEK

SEQ ID NO: 6 - protein, 569 aa, Streptomyces griseus
MGRTTLVEHDVPVEMRDGTVLRADVWRPADGPPSPAVLFRTPYGKSPLGLATLTPAQCVDRGYAAVVQDTRGRFGSEGEWAPLDWSREGPDGYDTVEWTAGQPWCDGNVAMAGTSYQAIVQWLAAMEKPPHLRAIAPTMSTSAPFDAEQLGGSLRLDHLTSWLGLTALEWVQRRAAAGDPVDGAVVAEVVQLLTTPDVPLRRWPLSTILDFEGFPGRLRDIFAGKVATVADYRLGEVGVPTFSVGGWYDVFSHGTIELHRAMRAQDPVAGRHELVVGPWVHSGQLPQVQGEVNTGPYGSAQGARLADLHLDFFDRHLRPSAADAGTAAGGDVRYFLFGDDTWHRAESWPPPEAVDSPWYLAGPEGGEEDGRLLPEPPQEGFGQDAFTYDPENPVPSHGGRVLQLGRLAAGPLNQAHLEARPDVLLYTSEPLTEPLDVVGRQRLRLRFGSDAPATAVVAKLTDVHPDGRSLLVAEASLRLDTGNGVPPDEPYDADLLLGDTAWRFAPGHRLRVHVTSSNFPHLDRHPNVPGPVGEAEHCRPARQTVWYGGPYESVLRLRVLPAPSTGGIA

SEQ ID NO: 7 - protein, 10 aa, Artificial Sequence
  synthetic peptide
AAAGGNNGGC

SEQ ID NO: 8 - protein, 7 aa, Artificial Sequence
  synthetic peptide
GGNNGGK

SEQ ID NO: 9 - protein, 6 aa, Artificial Sequence
  synthetic peptide
LAGGNK

SEQ ID NO: 10 - protein, 5 aa, Artificial Sequence
  synthetic peptide
AGGNK

SEQ ID NO: 11 - protein, 4 aa, Artificial Sequence
  synthetic peptide
GGNK

(19) United States
(12) Patent Application Publication
(10) Pub. No.: US 2023/0021352 A1
(43) Pub. Date: Jan. 26, 2023

(54) Improved Aminopeptidases for Single Molecule Peptide Sequencing

(71) Applicants: VIB VZW, Gent (BE); UNIVERSITEIT GENT, Gent (BE)

(72) Inventors: Nico Callewaert, Nevele (BE); Simon Devos, Sint-Michiels (BE); Sven Eyckerman, Nazareth (BE)

(21) Appl. No.: 17/783,595

(22) PCT Filed: Dec. 9, 2020
(86) PCT No.: PCT/EP2020/085250

§ 371 Date: Jun. 8, 2022

(30) Foreign Application Priority Data
Dec. 10, 2019 (GB) 1918108.0

(51) Int. Cl. C12Q 1/37; C12N 9/52; G01N 33/68

(52) U.S. Cl. CPC C12Q 1/37; C12N 9/52; G01N 33/6824

(57) ABSTRACT

The present invention relates to protein sequencing, more particularly the invention discloses improved aminopeptidases for single molecule protein sequencing and/or amino acid identification. Said aminopeptidases can enzymatically cleave off N-terminal amino acids and are highly suitable in a kinetics-based peptide sequencing approach. Based on the kinetics of the cleaving reaction or of the engagement between said aminopeptidases and peptide to be sequenced, information on the identity of the cleaved amino acids is provided.

### CROSS-REFERENCE TO RELATED APPLICATIONS

This application is a national phase entry under 35 U.S.C. § 371 of International Patent Application PCT/EP2020/085250, filed Dec. 9, 2020, designating the United States of America and published in English as International Patent Publication WO 2021/116163 on Jun. 17, 2021, which claims the benefit under Article 8 of the Patent Cooperation Treaty to United Kingdom Patent Application Serial No. 1918108.0, filed Dec. 10, 2019, the entireties of which are hereby incorporated by reference.

### FIELD OF THE INVENTION

The present invention relates to the field of protein sequencing. The invention discloses improved aminopeptidases particular useful in methods for single molecule protein sequencing.

### BACKGROUND

For both fundamental research and diagnostic purposes, there is a need for high throughput sequencing of single molecule peptides. Several concepts for next-generation protein sequencing have been proposes. In analogy with the DNA nanopore sequencing technology, it has for example been suggested to sequence peptides through (solid-state) nanopores (WO2014014347A1; WO2015126494A1). Extensive research is done on the engineering of nanopores that are able to translocate peptides and differentiate between amino acids or amino acid categories along the sequence (Kennedy et al. 2016 Nat Nanotechnol 11:968-976; Wilson et al. 2016 Adv Funct Mater 26:4830-4838). Another approach is based on an intelligent yet complicated process of converting the sequential order of amino acids of the peptide into a nucleic acid fragment (WO2017192633A1). This approach uses a battery of different oligonucleotide-labelled binders each recognizing different N-terminal amino acids. In a stepwise procedure of binding to and cleaving of amino acids, the oligo tags on the binders anneal and construct a nucleic acid molecule comprising the information of position and identity of amino acids of which the peptide is comprises. Said nucleic acid molecule can then be sequenced through one of the well validated DNA sequencing methods and decoded back to a peptide sequence. A third approach also uses amino acid binders but for direct identification of N-terminal amino acids. The methods gather protein sequence information by successive cycles of labeling the peptides' N-terminal amino acid, detecting the label and removal of the labelled N-terminal amino acid (WO2010065531A1; WO2012178023A1; WO2013112745A1; US20140273004A1). Removal can be obtained by a classic Edman degradation process or enzymatically using Edmanases (US20140273004A1). The disadvantage of this and previous methods is that for every amino acid a specific N-terminal amino acid binder should be used, increasing the complexity of the method.

The applicants of current application previously disclosed for the first time that the kinetics of the engagement between a N-terminal amino acid binder and the amino acid and/or the kinetics of the cleaving reaction of an aminopeptidase provides information on the identity of the N-terminal amino acid. By using only one or a limited number of non-selective, broad-spectrum N-terminal amino acid binders the number of reagents needed and thus the complexity of the method is highly reduced. Said method was demonstrated in WO2019063827A1 using a Thermus aquaticus aminopeptidase and a Trypanosoma cruzi cruzipain as N-terminal amino acid binders. However, these peptidases have some drawbacks. The T. aquaticus aminopeptidase for example consists of 2 domains. As a result, the peptide substrates cleavable by said enzyme are restricted to about 10 amino acids. The cruzipain on the other hand is not thermostable and can therefore not be used when secondary peptide structures need to be denatured.

### SUMMARY

To further optimize our kinetics-based peptide sequencing method, we have selected new aminopeptidases. These aminopeptidases are monomeric, single domain enzymes, are thermophilic or thermostable and are broad spectrum but with a preference towards certain N-terminal amino acids, thereby overcoming the above-mentioned problems.

Therefore, in a first aspect, the application provides an aminopeptidase selected from the list consisting of Aeromonas proteolytica aminopeptidase, Serratia marcescens aminopeptidase, Pyrococcus furiosus aminopeptidase, Lactobacillus helveticus X-prolyl dipeptidyl aminopeptidase, Streptomyces griseus X-prolyl dipeptidyl aminopeptidase and Streptomyces griseus aminopeptidase, coupled to an optical, electrical or plasmonic label for detecting said aminopeptidase. In one embodiment, said aminopeptidase is catalytically active and comprises an amino acid sequence that is at least 80% identical to and over the full length of SEQ ID No. 1-6. Also the use of said labelled aminopeptidase or the binding and/or cleavage kinetics of said labelled aminopeptidase is provided to obtain sequence information of a C-terminally immobilized polypeptide.

In a second aspect, a method of identifying or categorizing the N-terminal amino acid of a polypeptide immobilized on a surface via its C-terminus is provided, said method comprising:
a) contacting said surface immobilized polypeptide with at least one aminopeptidase suitable for binding and cleaving the N-terminal amino acid from said polypeptide;
b) measuring the residence time of said aminopeptidase on said N-terminal amino acid;
c) optionally allowing said aminopeptidase to cleave off said N-terminal amino acid;
d) comparing said measured residence time to a set of reference residence time values characteristic for said aminopeptidase and a set of N-terminal amino acids,
to identify or categorize said N-terminal amino acid,
   characterized by said aminopeptidase being selected from the list consisting of Aeromonas proteolytica aminopeptidase, Serratia marcescens aminopeptidase, Streptomyces griseus aminopeptidase and Pyrococcus furiosus aminopeptidase or Lactobacillus helveticus X-prolyl dipeptidyl aminopeptidase or Streptomyces griseus X-prolyl dipeptidyl aminopeptidase, more particularly said aminopeptidase is catalytically active and comprises an amino acid sequence that is at least 80% identical to and over the full length of SEQ ID No. 1-6.

In one embodiment, steps a) through d) or steps b) through d) are repeated one or more times. In another embodiment, wherein said residence time is measured optically, electrically or plasmonically. In another embodiment, the residence time of said aminopeptidase is measured for every binding event of said aminopeptidase to said N-terminal amino acid. In another embodiment, above methods are provided additionally including a step of determining the cleavage of said N-terminal amino acid by measuring an optical, electrical or plasmonical signal of the surface-immobilized polypeptide, wherein a difference in optical, electrical or plasmonical signal is indicative for cleavage of said N-terminal amino acid. In yet another embodiment, said methods further include a first step of polypeptide denaturation or include one or more of the steps in which polypeptide denaturing conditions are present. In particular embodiments, said polypeptide is immobilized on an active sensing surface, more particularly a gold surface or an amide-, carboxyl-, thiol- or azide-functionalized surface on which said polypeptide is chemically coupled.

In a third aspect, a kit of parts is provided comprising a surface for immobilization of peptides and an aminopeptidase selected from the list consisting of Streptomyces griseus aminopeptidase, Aeromonas proteolytica aminopeptidase, Serratia marcescens aminopeptidase and Pyrococcus furiosus aminopeptidase. In a particular embodiment, said kit further comprises a X-prolyl dipeptidyl aminopeptidase; more particularly a Lactobacillus helveticus X-prolyl dipeptidyl aminopeptidase or Streptomyces griseus X-prolyl dipeptidyl aminopeptidase.

### BRIEF DESCRIPTION OF THE FIGURES

The patent or application file contains at least one drawing, executed in color, Copies of this patent or patent application publication with color drawing(s) will be provided by the Office upon request and payment of the necessary fee.

FIG. 1. Kinematic monitoring of enzymatic degradation for single molecule peptide sequencing

FIGS. 2A and 2B. Leucine-p-nitroanilide aminopeptidase assay in the presence of denaturing agents. The degradation of Leu-pNA by the S. griseus aminopeptidase was monitored at A405 nm at different concentrations of methanol or acetonitrile (FIG. 2A) and urea (FIG. 2B).

FIGS. 3A and 3B. S. griseus aminopeptidase ‘on-time’ monitoring on single molecule, C-terminal immobilized peptides. (FIG. 3A) Single molecule peptide detection with TIRF microscopy. (FIG. 3B) S. griseus on-time monitoring.

FIG. 4. Expression of the different aminopeptidases in E. coli. S. griseus aminopeptidase (SGAP), S. marcescens aminopeptidase (SMAP), A. proteolytica aminopeptidase (APAP) and P. furiosus aminopeptidase (PFAP) were produced in E. coli BL21(DE3) and detected with western blot analysis.

FIG. 5. Activity of the E. coli expressed aminopeptidases at varying temperature. S. griseus aminopeptidase (SGAP), S. marcescens aminopeptidase (SMAP) and P. furiosus aminopeptidase (PFAP) were produced in E. coli BL21(DE3) and purified with IMAC. The activity of the purified aminopeptidases was monitored with leucine-p-nitroanilide, proline-p-nitroanilide and methionine-p-nitroanilide, respectively, at different temperatures.

FIGS. 6A and 6B. Percentage of uniquely identified peptides of the (FIG. 6A) complete human C-terminome and (FIG. 6B) human plasma C-terminome, after Trypsin, LysC or CysC protein cleavage, when either Met, Pro, Leu, Leu/Met or Leu/Met/Pro residues are correctly located in the sequence.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4^{th} ed., Cold Spring Harbor Laboratory Press, Plainsview, N.Y. (2012); and Ausubel et al., Current Protocols in Molecular Biology (Supplement 47), John Wiley & Sons, N.Y. (1999), for definitions and terms of the art. The definitions provided herein should not be construed to have a scope less than understood by a person of ordinary skill in the art.

In current application, Applicants disclose aminopeptidases for binding and cleaving N-terminal amino acids of C-terminal immobilized peptides. Said aminopeptidases are selected for improved compatibility with the single molecule peptide sequencing methods previously disclosed in WO2019063827A1. First, the aminopeptidases are monomeric and single-domain, with an accessible catalytic site that has minimal constraints in terms of peptide substrate length. Most aminopeptidases are either multimeric or have multiple domains. These features lead to a limited accessibility of the catalytic site. Only short, unstructured peptides, for example products of endoproteases, can then be processed. Furthermore, some aminopeptidases completely enclose peptide substrates before cleaving them. This is problematic for cleavage of surface-immobilized peptides. Second, the aminopeptidases have a preference towards certain N-terminal amino acids, however can bind to (and optionally cleave of) a broad range of N-terminal amino acids, preferably all N-terminal amino acids. Therefore, these aminopeptidases are considered to be ‘broad specific’ and provide a solution to the need of a plethora of different N-terminal amino acid binders. Third, the aminopeptidases are thermostable, thermophilic or solvent resistant. During processing, the peptide secondary structure should be denatured as much as possible to minimize its effect on catalytic efficiency. Working at higher temperature is one way to deal with this. Alternatively, denaturation can be achieved chemically. Aminopeptidases that are not able to withstand these harsh conditions are of limited use. Interestingly, most thermophilic enzymes can not only tolerate high temperatures but also tolerate higher concentrations of organic solvents (e.g. methanol, acetonitrile) and denaturing salts (e.g. ureum).

### Improved Aminopeptidases

The inventors of current application have selected aminopeptidases that can be implemented in the previously disclosed kinetic-based peptide sequencing method (WO2019063827A1). These aminopeptidases are Streptomyces griseus aminopeptidase (SGAP; UniProtKB-P80561) as depicted in SEQ ID No. 1, Aeromonas proteolytica aminopeptidase (APAP; UniProtKB-Q01693) as depicted in SEQ ID No. 2, Serratia marcescens aminopeptidase (SMAP; UniProtKB-032449) as depicted in SEQ ID No. 3 and Pyrococcus furiosus aminopeptidase (PFAP; UniProtKB-P56218) as depicted in SEQ ID No. 4. Aeromonas proteolytica aminopeptidase is also called Vibrio proteolyticus aminopeptidase.

These aminopeptidases are particularly suited for use in the methods of WO2019063827A1 (for detailed description see below), however their use is not limited to that. The aminopeptidases herein disclosed remove N-terminal amino acids and can therefore be used in the methods of US2014273004A1, U.S. Pat. No. 9,435,810B2, US20170052194A1 and WO2017192633A1 as well.

### Kinetics-Based Peptide Sequencing Methods of WO2019063827A1

The kinetics-based peptide sequencing methods as disclosed in WO2019063827A1 are characterized by a multiple step approach in which the N-terminal amino acids of C-terminally immobilized polypeptides are identified one by one. The methods comprise the steps of:
a) contacting a C-terminally immobilized polypeptide with a catalytically active aminopeptidase;
b) measuring the residence time of said aminopeptidase on the N-terminal amino acid of said polypeptide or alternatively measuring the k_{cat} value of said enzymatic reaction;
c) identifying or categorizing said N-terminal amino acid by said residence time or said k_{cat} value; and
d) repeating the steps a) through c) one or more times.

For current application said methods are provided wherein said catalytically active aminopeptidase is the aminopeptidase selected from the list consisting of Streptomyces griseus aminopeptidase, Aeromonas proteolytica aminopeptidase, Serratia marcescens aminopeptidase and Pyrococcus furiosus aminopeptidase. In one embodiment, said catalytically active aminopeptidase is fused to an optical, electrical or plasmonic label for detecting said aminopeptidase.

In general, an enzyme's specificity for a particular substrate under particular environmental conditions can be quantified by the specificity constant k_{cat}/K_{M}. k_{cat} is the turnover number, the number of substrate molecules each enzyme site converts to product per unit of time, or the number of productive substrate to product reaction per catalytic center and per unit of time. K_{M} is defined as the substrate concentration required for the enzyme to reach half of its maximal velocity under the conditions required for valid steady state enzyme kinetics measurements, well known in the art. When distinguishing two enzyme substrates A and B, based on the rate of conversion of these substrates to products, relations of this type hold:

$$\frac{{v}_{A}}{{v}_{B}} = \frac{{dP}_{A}}{{dP}_{B}} = \frac{( {V}_{{A}_{max}} / {K}_{{M}_{A}} ) [ A ]}{( {V}_{{B}_{max}} / {K}_{{M}_{B}} ) [ B ]} = \frac{( {k}_{A} / {K}_{{M}_{A}} ) [ A ]}{( {k}_{B} / {K}_{{M}_{B}} ) [ B ]}$$

with v velocity, and [A] the concentration of A.

Consequently, information on the identity of different substrates of an enzyme can be gained from conversion velocity measurements of these substrates by the enzyme. Under conditions of equal substrate concentrations, relative velocities are determined by k_{cat} and K_{M}. When observing a single substrate molecule, once the enzyme is added, the time required to form a product molecule is governed by k_{cat}. Hence, in single molecule observations, information on the identity of the substrate can be gained from the “on-time” or residence time of the enzyme on the substrate. This information can further be complemented by engineering the substrates and/or the enzyme such that catalytically productive engagements of the enzyme and substrate can be distinguished from non-productive ones. Thus “on-time” or tₒₙ as used herein refers to the residence time of the enzyme on the substrate, the contact time of the enzyme solution with the substrate or more particularly to the inverse of k_{cat}, which is well known in the art. From here on “on-time” and “residence time” will be used interchangeably and can refer to the time of one enzyme molecule acting on one peptide molecule until cleavage occurs or to the time required for multiple enzyme molecules acting sequentially on the peptide molecule until cleavage occurs.

Crucial for the methods is that the polypeptide to be sequenced or of which the N-terminal amino acid is to be identified or categorized is immobilized through the moiety which is most C-terminal of the polypeptide or through the moiety C-terminal of the scissile bond. The polypeptide is thus attached to the surface of the application with its C-terminus or with a moiety along the peptide's structure, C-terminal to the scissile bond (e.g. with a cysteine's thiol function through e.g. maleimide chemistry or gold-thiol bonding, well known in the art). “Scissile bond” as used herein refers to the covalent chemical bond to be cleaved by one of the aminopeptidases of the application. The peptide may be immobilized on any suitable surface (see WO2019063827A1).

The observation that “on-time” of an enzyme on a substrate can be used to identify said substrate holds especially true for aminopeptidases. Peptidases generally operate through a two-step mechanism. First, during an acylation reaction the N-terminal moiety of the peptide (for aminopeptidases) or the C-terminal moiety of the peptide (for carboxypeptidases) is cleaved off and covalently linked to the peptidase. Second, in a deacylation reaction the enzyme releases the cleaved amino acid.

An aminopeptidase gains its specificity for particular (groups of) amino acids through a stereo-electronic fit with the transition state of the acylation reaction, impacted among others by the nature of the side chain(s) of the substrate to the N-terminus of the scissile bond. Typically, aminopeptidases have much less binding interactions with the peptide moiety to the C-terminus of the scissile bond, and will thus rapidly dissociate from the peptide (or from the surface to which the peptide was bound) upon the reaction rate-determining acylation or hydrolysis step. If a peptide is immobilized C-terminally from the scissile peptide bond that is cleaved by the peptidase, then upon the acylation reaction, the N-terminal amino acid will be covalently linked to the enzyme in the case of a serine or cysteine peptidase, or will be non-covalently bound to the enzyme in case of directly hydrolyzing peptidases, whereas the C-terminal moiety will remain conjugated to the surface on which the peptide was immobilized. Consequently, for selected aminopeptidases, the residence time or the “on-time” on the surface-immobilized peptide substrate is a correlate for the rate of the acylation or hydrolysis step, and hence for the nature of the moiety N-terminal to the scissile bond. The “on-time” of an aminopeptidase can in this case easily be determined by molecularly labelling said aminopeptidase. As such the molecular label acts as a proxy for the “on-time” of the aminopeptidase and thus for the identity of the N-terminal amino acid that is cleaved off by said aminopeptidase. In a particular embodiment of this application, said aminopeptidase can be optically, fluorescently, electrically or plasmonically labelled (see later). Alternatively, also a solution of aminopeptidase molecules can be contacted with the peptide substrate and the residence time/on-time is then measured until the N-terminal amino acid (or a derivative thereof) is cleaved off. The overall residence time of the enzyme in contact with the substrate is then measured until such cleavage event, and this value correlates with the inverse of k_{cat} of the enzyme for the particular N-terminal amino acid (derivative) on the peptide substrate under the conditions that are used.

For carboxypeptidases from the group of cysteine and serine proteases, the situation is different. More precisely, in case of said carboxypeptidases, the enzyme stays covalently bound to the immobilized peptide moiety after cleaving off the C-terminal amino acid. The carboxypeptidase will not dissociate from the peptide upon the acylation step and it's “on-time” value on the peptide on the immobilization surface will be determined by the rate of the deacylation (hydrolysis) step. The latter hydrolysis step is much less or not informative for the nature of the C-terminal amino acid (which was already released in the solvent during the acylation step).

The aminopeptidases disclosed herein are thermophilic and/or solvent resistant. This requirement is based on two observations. First, by adjusting the reaction conditions during the protein sequencing procedure (e.g. temperature, pH, solvents, . . . ) the “on-time” values of aminopeptidases can be fine-tuned to differentiate more between the “on-time” value for amino acid X and the “on-time” value for amino acid Y. To maintain the enzymatic activity in less optimal physiological conditions, the aminopeptidase should be thermophilic, thermostable and/or solvent resistant. Interestingly, it was found that most thermophilic aminopeptidases tolerate solvents as well.

Second, it is advisable to include a protein denaturation step in the protein sequencing procedure. Proteins are amino acid polymers. Once genetic information is translated by the ribosomes into a protein and the subsequent post-translational modification process has been completed, the protein begins to fold (sometimes spontaneously and sometimes with enzymatic assistance), curling up on itself so that hydrophobic elements of the protein are buried deep inside the structure and hydrophilic elements end up on the outside. The final shape or structure of a protein determines how it interacts with its environment. As such, proteins have a primary structure (i.e. the sequence of amino acids held together by covalent peptide bonds), secondary structure (i.e. regular repeating patterns such as alpha-helices and beta-pleated sheets), tertiary structure (i.e. covalent interactions between amino acid side-chains such as disulfide bridges between cysteine groups) and quaternary structure (i.e. protein sub-units that interact with each other). However, for the peptide sequencing methods disclosed herein and in WO2019063827A1, the protein and its N-terminal amino acid should be accessible for the aminopeptidases of the application and preferably the protein is immobilized in a linear configuration. Therefore, in various embodiments, the protein to be sequenced is to be denatured. Denaturation is a process in which proteins lose the quaternary structure, tertiary structure and secondary structure which is present in their native state, but the peptide bonds of the primary structure between the amino acids are left intact. Protein denaturation can be achieved by applying external stresses or compounds such as a strong acid or base, a concentrated inorganic salt, an organic solvent (e.g., alcohol or chloroform), radiation or heat. It goes without saying that the aminopeptidases used in such procedure should be thermophilic and/or solvent resistant.

The aminopeptidases herein disclosed are particularly useful in the methods of WO2019063827A1. Hence, in one aspect, a method is provided of identifying or categorizing the N-terminal amino acid of a surface-immobilized polypeptide, said method comprising:
a) contacting said surface immobilized polypeptide with at least one of the aminopeptidases herein disclosed for binding and cleaving the N-terminal amino acid from said polypeptide;
b) measuring the residence time of said at least one aminopeptidase on said N-terminal amino acid;
c) comparing said measured residence time to a set of reference residence time values characteristic for said at least one aminopeptidase and a set of N-terminal amino acids;
   to identify or categorize said N-terminal amino acid.

Also a method is provided of obtaining sequence information of a surface-immobilized polypeptide, said method comprising:
a) contacting said surface-immobilized polypeptide with at least one of the aminopeptidases herein disclosed for binding and cleaving the N-terminal amino acid from said polypeptide;
b) measuring the residence time of said at least one aminopeptidase on the N-terminal amino acid of said surface-immobilized polypeptide;
c) identifying or categorizing said N-terminal amino acid by comparing said measured residence time to a set of reference residence time values characteristic for said at least one aminopeptidase and a set of N-terminal amino acids;
d) allowing said at least one aminopeptidase to cleave off said N-terminal amino acid;
e) repeating steps a) through d) one or more times.

In said methods, said residence time is measured optically, electrically or plasmonically (see later).

In specific embodiments, said step of measuring the residence time of said aminopeptidase on said N-terminal amino acid in above methods is measuring the residence time of said aminopeptidase on the N-terminal amino acid until cleavage of the N-terminal amino acid of said surface-immobilized polypeptide.

Alternatively, the enzyme tₒₙ/t_{off} can be monitored. The tₒₙ/t_{off} ratio will increase when the affinity for the N-terminal amino acid is higher (low K_{M}), and vice versa. On the other hand, the total time until a cleavage event occurs will increase when the turnover rate is lower (low k_{cat}), and vice versa.

As already discussed herein, the polypeptides immobilized on a surface should be denatured so that the N-terminus is freely accessible (in case the polypeptide is immobilized through its C-terminus) for enzymatic cleavage but also to avoid steric hindrance or interference of said cleavage. Therefore, the methods of current application are also provided including a first step of polypeptide denaturation. In various embodiments of this application, the methods herein described for identifying or categorizing N-terminal amino acids from a C-terminally immobilized polypeptide or for obtaining sequence information from said polypeptide are methods executed on a single molecule level.

For single molecule measurements, it is envisaged that polypeptides from the methods of current application are immobilized on an active sensing surface. In particular embodiments, said active sensing surface is either a gold surface or an amide-, carboxyl-, thiol- or azide-functionalized surface on which said polypeptide is chemically coupled.

Multiple Measurements of Residence Time and Combined Use with Non-Cleaving Binders

In alternative embodiments, the aminopeptidases herein disclosed and useful in the methods of WO2019063827A1 cleave the N-terminal amino acids only after several rounds of binding and unbinding of the N-terminal amino acids. Every residence time of said aminopeptidases will be informative to determine the residence time until the N-terminal amino acid has been cleaved off, and may help to identify the N-terminal amino acid. In order to detect the time point of change of the identity of the N-terminal amino acid by the aminopeptidase and to predict the N-terminal amino acids more accurately in a single molecule set-up, it is recommended to have multiple measurements for every N-terminal amino acid. This can be achieved by using aminopeptidases that will dock to (association) and undock from (dissociation) the N-terminal amino acid several times before the actual cleavage will occur. It is thus also envisaged that the step of measuring the residence time of catalytically active aminopeptidases in the methods of the application implies the measuring of multiple residence times of said aminopeptidases before said aminopeptidase cleaves the N-terminal amino acid. Alternatively phrased, the residence time of said catalytically active aminopeptidase can be measured for every binding event of said aminopeptidase to said N-terminal amino acid. The above is demonstrated in WO2019063827A1. In particular embodiments, the methods disclosed in current application are provided wherein the aminopeptidase used in the enzymatic cleavage of the N-terminal amino acids on average has at least 3, at least 4, at least 5, at least 6, at least 7, at least 8, at least 9, at least 10, at least 20 or at least 50 association/dissociation cycles in the time window required for said aminopeptidase to cleave an N-terminal amino acid. This means that at least 3, at least 4, at least 5, at least 6, at least 7, at least 8, at least 9, at least 10, at least 20 or at least 50 cleavage-unproductive association/dissociation cycles occur in between cleavage-productive ones.

Also provided are the methods of current application wherein said surface-immobilized polypeptide is additionally contacted with one or more terminal amino acid binding proteins, wherein the kinetics of the binding events of said one or more binding proteins to said terminal amino acid identify said terminal amino acid. The possibility of using binding specificities of N-terminal amino acid binding proteins to gather information of the substrate is theoretically demonstrated by Rodrigues et al (2018, PLoS ONE 14(3): e0212868). The additional use of said non-cleavable binders (next to a catalytically active aminopeptidase) in the method of current application can provide additional information in order to predict or identify N-terminal amino acids with a higher accuracy in single molecule experiments. In particular embodiments, said non-cleavable binders have at least 3, at least 4, at least 5, at least 6, at least 7, at least 8, at least 9, at least 10, at least 20 or at least 50 association/dissociation cycles with the N-terminal amino acid in the time window required for one of the aminopeptidases of the application to cleave said N-terminal amino acid.

### Detection of Cleavage

As in WO2019063827A1, one of the additional parts of the methods of the application is that the cleavage of the terminal amino acid is to be detected or confirmed. Hence also provided herein are the methods of current application, additionally including a step of determining the cleavage of said terminal amino acid by measuring an optical, electrical or plasmonical signal of the surface-immobilized polypeptide, wherein a difference in optical, electrical or plasmonical signal is indicative for cleavage of said terminal amino acid. Indeed, immobilized peptides with a free N-terminus have several properties which are utilized to determine when an N-terminal amino acid has been cleaved off by the cleaving-inducing agents of the present application.

Methods of detecting the cleavage are as provided in WO2019063827A1 described on page 29 line 23 until page 32 line 5.

In most particular embodiments of current application, the method as described herein are performed in protein denaturing conditions. Said protein denaturing conditions are obtained by high temperature and by the presence of solvents. In particular embodiments, said high temperature is a temperature between 40° C. and 120° C. or between 50° and 110° C. or between 60° C. and 100° C. or between 70° C. and 90° C. In particular embodiments, said solvent is selected from the list consisting of acetic acid, trichloroacetic acid, sulfosalicyclic acid, sodium bicarbonate, ethanol, alcohol, cross-linking agents such as formaldehyde and glutaraldehyde, chaotropic agents such as urea, guanidinium chloride, lithium perchlorate, and agents that break disulfide bonds such as 2-mercaptoethanol, dithiothreitol, or tris(2-carboxyethyl)phosphine. Most particularly said solvent is acetonitrile, ethanol or methanol.

### Measuring Residence Times

To detect the presence of an aminopeptidase on the N-terminal amino acids of C-terminally immobilized peptides and thus to measure or determine the “on-time” values or residence times of the aminopeptidase, two labelling options can be selected. First, the polypeptides to be sequenced can be labelled for example through their N-terminal amino acids or via internal amino acids. The procedure is described in WO2019063827A1 page 21 lines 4-24. Second, the aminopeptidase itself can be labeled. This is explained in WO2019063827A1 on page 21 line 26 until page 24 line 8. It must be clear that the nature of labelling and consequently detection is not vital to the invention, as long as the “on-time” or the residence time of the aminopeptidases can be detected and determined.

In one aspect, current application provides a labelled protein comprising an aminopeptidase more particularly a catalytically active aminopeptidase selected from the list consisting of Streptomyces griseus aminopeptidase, Aeromonas proteolytica aminopeptidase, Serratia marcescens aminopeptidase and Pyrococcus furiosus aminopeptidase, and an optical, electrical or plasmonic label for detecting said aminopeptidase. Alternatively phrased, an aminopeptidase selected from the list consisting of Streptomyces griseus aminopeptidase, Aeromonas proteolytica aminopeptidase, Serratia marcescens aminopeptidase and Pyrococcus furiosus aminopeptidase coupled to an optical, electrical or plasmonic label for detecting said aminopeptidase is provided. In one embodiment, “coupled to” means covalently or non-covalently bound to. In another embodiment, the labelled aminopeptidase is produced through recombinant DNA technologies in which a fusion protein is formed comprising the aminopeptidase and a genetically encoded or a molecular label. In a particular embodiment, said genetically encoded or molecular label is an optical label, even more particularly a fluorescent or luminescent protein.

As explained earlier, aminopeptidases selected and used herein are the proteins depicted in SEQ ID No. 1-4. However, it goes without saying that the aminopeptidases should not be 100% identical to said sequences to be useful in the methods herein disclosed. Indeed, as long as the binding properties and the catalytical activity of said aminopeptidases are not changed, aminopeptidases that differ to SEQ ID No. 1-4 in several amino acids or even short fragments will be as suitable. Therefore, current application discloses catalytically active aminopeptidases with an amino acid sequence that is at least 80%, at least 85%, at least 90%, at least 91%, at least 92%, at least 93%, at least 94%, at least 95%, at least 96%, at least 97%, at least 98% or at least 99% identical to SEQ ID No. 1, 2, 3 or 4. Said identity is calculated over the full length of the SEQ ID No. 1-4 sequences.

In a most particular embodiment, the Streptomyces griseus aminopeptidase is SEQ ID No. 1, Aeromonas proteolytica aminopeptidase is SEQ ID No. 2, Serratia marcescens aminopeptidase is SEQ ID No. 3 and Pyrococcus furiosus aminopeptidase is SEQ ID No. 4. All aminopeptidases disclosed herein are also provided as coupled to an optical, electrical or plasmonic label for detecting said aminopeptidase.

In another aspect, the application also provides the use of any of the aminopeptidases, labelled aminopeptidases or fusion proteins herein disclosed for obtaining sequence information of a peptide, polypeptide or protein or for categorizing or identifying one or more amino acids of said peptide, polypeptide or protein. Also the use of the binding and/or cleavage kinetics of any of the aminopeptidases, labelled aminopeptidases or fusion proteins herein disclosed is provided for obtaining sequence information of a peptide, polypeptide or protein or for categorizing or identifying one or more amino acids of said peptide, polypeptide or protein. In one embodiment, said peptide, polypeptide or protein is immobilized on a surface via its C-terminus. “Categorizing” as used herein refers to catalogue an amino acid in a particular group for example but without the purpose of being limited: aromatic amino acids, non-aromatic amino acids, hydrophobic amino acids, positively charged amino acids, negatively charged amino acids, and small amino acids.

In yet another aspect, the application also provides a kit of parts comprising a surface for immobilization of a peptide, polypeptide or protein and an aminopeptidase selected from the list consisting of Streptomyces griseus aminopeptidase, Aeromonas proteolytica aminopeptidase, Serratia marcescens aminopeptidase and Pyrococcus furiosus aminopeptidase.

The peptide, polypeptide or protein to be sequenced may be immobilized on a surface prior to contact with the aminopeptidase. Therefore, the application also provides a kit of parts comprising a surface-immobilized peptide, polypeptide or protein and an aminopeptidase selected from the list consisting of Streptomyces griseus aminopeptidase, Aeromonas proteolytica aminopeptidase, Serratia marcescens aminopeptidase and Pyrococcus furiosus aminopeptidase. In one embodiment, the aminopeptidase is one selected from any aminopeptidase disclosed herein, more particularly from this list consisting of SEQ ID No. 1-4. In another embodiment, the aminopeptidase is one of the above described labelled aminopeptidases or fusion proteins. In another embodiment, the kit of parts is provided comprising a surface-immobilized peptide, polypeptide or protein and an aminopeptidase comprising an amino acid sequence that is at least 80%, at least 85%, at least 90%, at least 91%, at least 92%, at least 93%, at least 94%, at least 95%, at least 96%, at least 97%, at least 98% or at least 99% identical to SEQ ID No. 1, 2, 3 or 4. In a particular embodiment, said identity is calculated over the full length of the SEQ ID No. 1-4 sequences.

“Surface” as used herein is a synonym for carrier or layer. The surface or layer of current application is suitable to use in the detection of molecular labels, electrochemical signals, electromagnetic signals, plasmon related events. Said molecular label can be an optical (comprising but not limited to luminescent and fluorescent labels) or electrical (comprising but not limited to potentiometric, voltametric, coulometric labels) label.

Said layer can also be a multilayer, i.e. a layer that comprises several layers. In case of a multilayer, at least one layer should allow suitable detection of said molecular labels or said electrochemical, electromagnetic or plasmon related events. Therefore, according to particular embodiments, the surface is an active sensing surface. Hence, the surface immobilized polypeptide of said method of sequencing a surface-immobilized polypeptide at single molecule level is a polypeptide immobilized on an active sensing surface. In more particular embodiments, said active sensing surface is either a gold surface or an amide-, carboxyl-, thiol- or azide-functionalized surface on which the polypeptide of said method is chemically coupled. In other particular embodiments, said carrier is a nanoparticle, a nanodisk, a nanostructure, a chip. In most particular embodiments, said surface is a self-assembled monolayer (SAM).

### X-Prolyl Dipeptidyl Aminopeptidases

In the methods disclosed herein, the aminopeptidases of current application can have limited processability towards a N-terminal amino acid X that is followed by a proline. Due to proline's unique structure, the peptide bond between any N-terminal amino acid that is followed by a proline (also referred to as a X-pro peptide bond) is often resistant to most (amino)peptidases (Walter et al 2018 Mol Cel Biochem 30). However, this binding can be cleaved by X-prolyl dipeptidyl aminopeptidases releasing the N-terminal amino acid X together with the proline. In order to overcome a premature stop during the sequencing methods herein disclosed because of the limited processability of the X-pro binding by the aminopeptidases of current application, a X-prolyl dipeptidyl aminopeptidase can be added in the methods of the application.

In one aspect, the application provides an X-prolyl dipeptidyl aminopeptidase selected from the list consisting of Lactobacillus helveticus X-prolyl dipeptidyl aminopeptidase (UniProtKB-A0A0C5KX33) and Streptomyces griseus X-prolyl dipeptidyl aminopeptidase. These X-prolyl dipeptidyl aminopeptidase have been selected because of their thermostability. In one embodiment, said X-prolyl dipeptidyl aminopeptidase is catalytically active and comprises an amino acid sequence that is at least 80%, at least 85%, at least 90%, at least 91%, at least 92%, at least 93%, at least 94%, at least 95%, at least 96%, at least 97%, at least 98%, at least 99% or 100% identical to and over the full length of SEQ ID No. 5 (Lactobacillus helveticus X-prolyl dipeptidyl aminopeptidase) or SEQ ID No. 6 (Streptomyces griseus X-prolyl dipeptidyl aminopeptidase). In another embodiment, said X-prolyl dipeptidyl aminopeptidase is coupled to an optical, electrical or plasmonic label for detecting said aminopeptidase.

In another aspect, the methods of the application are provided further comprising a step of contacting the surface immobilized polypeptide with an X-prolyl dipeptidyl aminopeptidase suitable for releasing an N-terminal amino acid attached to proline. In one embodiment, said X-prolyl dipeptidyl aminopeptidase is labelled such that its binding to the N-terminal amino acid can be differentially determined or distinguished from the binding of one of the other labelled aminopeptidases from the application.

In yet another aspect, the kit of parts herein disclosed is provided further comprising a X-prolyl dipeptidyl aminopeptidase, more particularly one of the X-prolyl dipeptidyl aminopeptidases herein disclosed.

### Definitions

As used herein, the terms “peptide” and “polypeptide” are used interchangeably and refer to a polymeric form of amino acids of any length, which can include coded and non-coded amino acids, natural and non-natural amino acids, chemically or biochemically modified or derivatized amino acids, and polypeptides having modified peptide backbones. As used herein “peptides” or “polypeptides” are shorter than the full-length protein from which they derive and are formed for example but without the purpose of limiting by trypsin or proteinase K protein digestion. In particular embodiments, said peptides or polypeptides have a length between 20 and 500, or between 25 and 200 or between 30 and 100 amino acids or have a length of less than 500, less than 250, less than 200, less than 150, less than 100 or less than 50 amino acids. In any case, “peptide” or “polypeptide” comprises at least 2, at least 3, at least 4, at least 5, at least 6, at least 7, at least 8, at least 9, at least 10 or at least 20 amino acids.

“Single-molecule” as used in single molecule manner or at a single molecule level or in single molecule experiment refers to the investigation of the properties of individual molecules. Single-molecule studies may be contrasted with measurements on an ensemble or bulk collection of molecules, where the individual behavior of molecules cannot be distinguished, and only average characteristics can be measured.

“Immobilization on a surface” as used herein refers to the attachment of one or more polypeptides to an inert, insoluble material for example a glass surface resulting in loss of mobility of said polypeptides. For the methods disclosed in current application, immobilization allows the polypeptide(s) to be held in place throughout the sequencing of the polypeptide or identifying or categorizing the N-terminal amino acid of said polypeptide. The N-terminus should thus be freely accessibly, hence the polypeptide should be immobilized through its C-terminus. Moreover, proteins immobilized onto surfaces with high density allow the usage of small amount of sample solution. Many immobilization techniques have been developed in the past years, which are mainly based on the following three mechanisms: physical, covalent, and bioaffinity immobilization (Rusmini et al 2007 Biomacromolecules 8: 1775-1789; U.S. Pat. No. 6,475,809; WO2001040310; U.S. Pat. No. 7,358,096; US20100015635; WO1996030409). In particular embodiments, polypeptides are immobilized on glass surfaces as described in WO2019063827A1.

“Thermophilic” as used herein refers to “increased temperature tolerant”, more precisely to an organism or enzyme among others that thrives or maintains its activity at relatively high temperatures between 40 and 122° C. In particular embodiments, the aminopeptidases for the uses and methods of current application have optimal peptidase activity in a temperature range of 40° C. and 100° C. or of 40° C. and 80° C. or of 50° C. and 70° or of 60° C. and 80° C. In other particular embodiments, the aminopeptidases of the application maintain their enzymatic activity in the presence of solvents as acetic acid, trichloroacetic acid, sulfosalicyclic acid, sodium bicarbonate, ethanol, alcohol, cross-linking agents such as formaldehyde and glutaraldehyde, chaotropic agents such as urea, guanidinium chloride or lithium perchlorate, agents that break disulfide bonds such as 2-mercaptoethanol, dithiothreitol, or tris(2-carboxyethyl)phosphine.

“Aminopeptidase” as used herein refers to an enzyme that catalyzes the cleavage of amino acids from the amino terminus (N-terminus) of protein or peptide substrates. They are widely distributed throughout the animal and plant kingdoms and are found in many subcellular organelles, in cytosol, and as membrane components. Aminopeptidase are classified by 1) the number of amino acids cleaved from the amino terminus of substrates (e.g. aminodipeptidases remove intact amino terminal dipeptides, aminotripeptidases catalyze the hydrolysisis of amino terminal tripeptides), 2) the location of the aminopeptidase in the cell, 3) the susceptibility to inhibition by bestatin, 4) the metal ion content and/or residues that bind the metal to the enzyme, 5) the pH at which maximal activity is observed and 6) which is most relevant for this application by the relative efficiency with which residues are removed (Taylor 1993 FASEB J 7:290-298). Aminopeptidases can have a broad or a small substrate specificity. The improved aminopeptidase of this application are broad substrate specificity aminopeptidases.

An “X-prolyl dipeptidyl aminopeptidase” as used herein refers to an aminopeptidase that hydrolyzes peptides after proline.

“Catalytically active” means that the aminopeptidase is a fully functional catalytic enzyme. This in contrast to catalytically dead aminopeptidases that have been engineered to bind N-terminal amino acids but without cleaving said N-terminal amino acids, e.g. in WO20140273004.

As used herein, the terms “identical”, “similarity” or percent “identity” or percent “similarity” or percent “homology” in the context of two or more polypeptide sequences, refer to two or more sequences or subsequences that are the same or have a specified percentage of amino acid residues that are the same (e.g., 75% identity over a specified region) when compared and aligned for maximum correspondence over a comparison window or designated region as measured using sequence comparison algorithms or by manual alignment and visual inspection. Preferably, the identity exists over a region that is at least about 25 amino acids in length, or more preferably over a region that is 50-100 amino acids, even more preferably over a region that is 100-500 amino acids or even more in length.

The term “sequence identity” or “sequence homology” as used herein refers to the extent that sequences are identical on an amino acid by amino acid basis over a window of comparison. Thus, a “percentage of sequence homology” is calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity. A gap, i.e., a position in an alignment where a residue is present in one sequence but not in the other is regarded as a position with non-identical residues. Determining the percentage of sequence homology can be done manually, or by making use of computer programs that are available in the art. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyses is publicly available through the National Center for Biotechnology Information (http://www.ncbi.nlm.nih.gov/). In particular embodiments, the window of comparison to determine the sequence identity of two or more polypeptides (such as aminopeptidases) is the full length protein sequence.

The following examples are intended to promote a further understanding of the present invention. While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### EXAMPLES

### Example 1. Improved Aminopeptidases for Single Molecule Peptide Sequencing

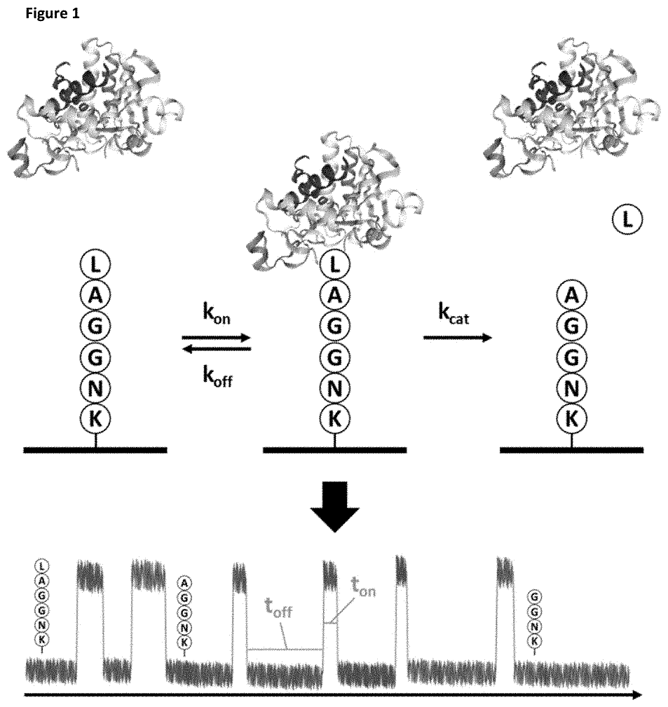

The single molecule peptide sequencing concept entails the use of active aminopeptidases that continuously bind and cleave the N-terminal amino acid of C-terminal immobilized peptides. Both amino acid affinity (K_{M}) and amino acid cleavage (k_{cat}) depends heavily on the identity of the N-terminal amino acid, with specificity constant values (k_{cat}/K_{M}) spanning several orders of magnitude (as described in WO2019063827A1). The time of the enzyme on the N-terminal amino acid between docking and undocking (herein referred to as the on-time or tₒₙ) can be monitored on single molecule peptide substrates over time (FIG. 1). The total time until a cleavage event occurs will increase (high tₒₙ) when the turnover rate is lower (low k_{cat}), and vice versa. On the other hand, when the affinity for the N-terminal amino acid is high (low K_{M}), the tₒₙ/t_{off} ratio will increase.

In order to optimally execute the peptide sequencing methods of WO2019063827A1, a selection of aminopeptidases for improved compatibility with said methods was performed. First, the aminopeptidases which are monomeric, single-domain enzymes, with an accessible catalytic site that has minimal constraints in terms of peptide substrate length were selected. Most aminopeptidases are either multimeric or have multiple domains, that leads to a limited accessibility of the catalytic site. Only short, unstructured peptides can be processed that are usually the product of endoproteases. Second, broad spectrum aminopeptidases with still a preference towards certain N-terminal amino acids were selected. A differential preference is particularly desirable for the methods of WO2019063827A1. Third, the aminopeptidases were selected for their thermostability or thermophilic characteristics. During processing, the peptide secondary structure should be denatured as much as possible to minimize its effect on catalytic efficiency. Working at higher temperature would be one way to deal with this. But usually thermophilic enzymes can also tolerate higher concentrations of organic solvents (e.g. methanol, acetonitrile) and denaturing salts (e.g. ureum).

Based on these criteria, a selection of four aminopeptidases was obtained: Streptomyces griseus aminopeptidase, Aeromonas proteolytica aminopeptidase, Serratia marcescens aminopeptidase and Pyrococcus furiosus aminopeptidase. All four aminopeptidases bind and cleave a broad spectrum of amino acids yet have a preference for one or more amino acids. While the S. griseus and A. proteolytica aminopeptidases have a preference for leucine, the S. marcescens aminopeptidase has a preference for proline and that of P. furiosus for methionine.

In order to monitor the binding and cleaving of the aminopeptidase(s) on the immobilized peptide substrates, a detectable tag is attached to the enzyme. These tags are conjugated either directly on the aminopeptidase using site-specific labeling on an N-terminal cysteine added to the protein, or the aminopeptidases are expressed as fusion protein (e.g. a VHH) where the tag is conjugated onto the fused protein, or the fused protein is on its own detectable (e.g. fluorescent protein).

### Example 2. Tolerance to Denaturing Conditions

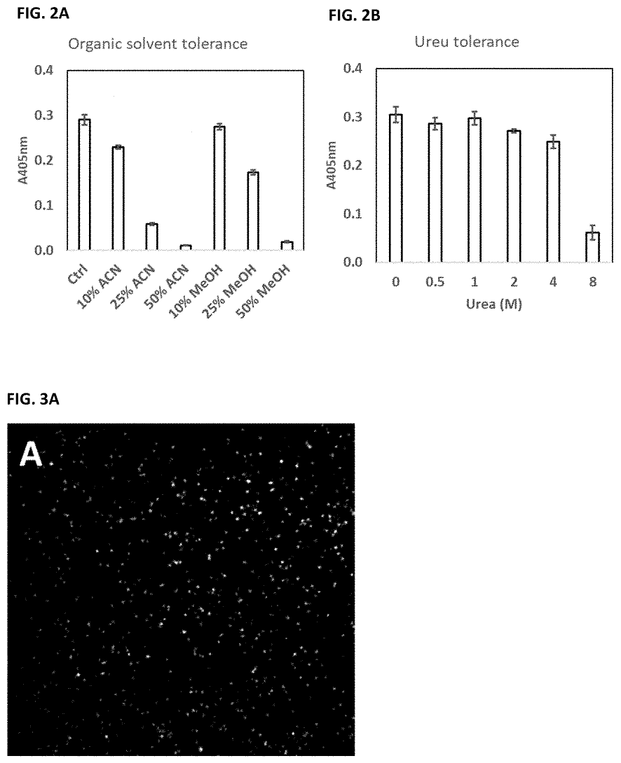

An aminopeptidase assay was performed with L-leucine-p-nitroaniline in PBS buffer containing different concentrations of organic solvent (methanol or acetonitrile) or urea (1.2 mM L-leucine-p-nitroaniline, 5 ng/μl aminopeptidase, 1 mM CaCl₂)) The mixture was incubated for 30 min at 30° C., after which the absorbance at 405 nm was measured. FIGS. 2A and 2B provides a representative situation of the improved aminopeptidase herein disclosed. The S. griseus aminopeptidase is stable in the presence of 10% acetonitrile (ACN), 10% methanol (MeOH) or 4 M urea (FIGS. 2A and 2B).

### Example 3. On-Time Measurements on Immobilized Peptides

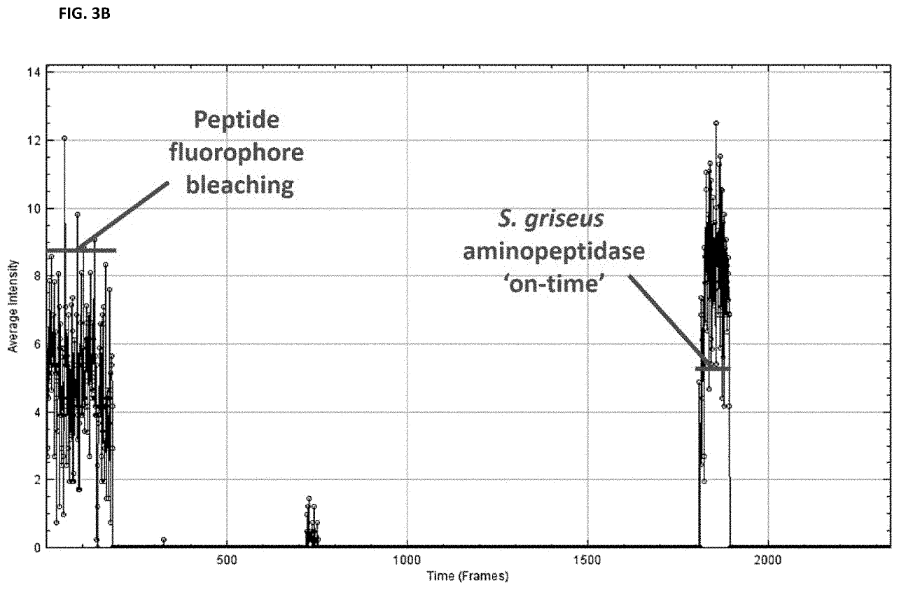

The fluorescent, synthetic peptide AAAGGNNGGC(DyLight650)GGNNGGK(dbco)G (1 nM) was immobilized on an azide-functionalized glass surface according to the methods described in WO2019063827A1 (Example 1). The immobilized single molecule peptides were then detected with TIRF microscopy (FIG. 3A). Single molecules were identified by a single drop in signal intensity during bleaching. After the peptide-conjugated fluorophores were bleached, sulfo-Cy5-labeled S. griseus aminopeptidase (100 pM) was added, and the peptide ‘on-time’ was monitored (FIG. 3B).

### Example 4. Expression of the Improved Aminopeptidases in E. coli

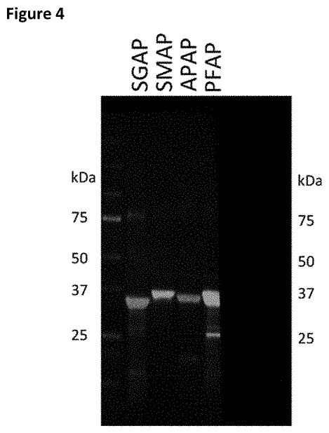

S. griseus aminopeptidase (SGAP), S. marcescens aminopeptidase (SMAP), A. proteolytica aminopeptidase (APAP) and P. furiosus aminopeptidase (PFAP) were produced in E. coli BL21(DE3) in 100 ml LB medium. Cultures were grown at 37° C. in shake flasks until an OD₆₀₀ of 0.8-1.0 was reached. Then 1 mM IPTG was added to induce protein expression, and cultures were allowed to grow further at 28° C. overnight. Cells were collected via centrifugation, and lysed in 50 mM Tris-HCl/10 mM imidazole (pH 8) through sonication. Either the crude lysate, or the NiNTA-purified protein fraction, was separated on SDS-PAGE and finally the aminopeptidases were detected via western blot analysis using an anti-His-Tag antibody carrying DyLight800 fluorophores (FIG. 4).

### Example 5. Activity of the E. coli Expressed Aminopeptidases at Varying Temperature

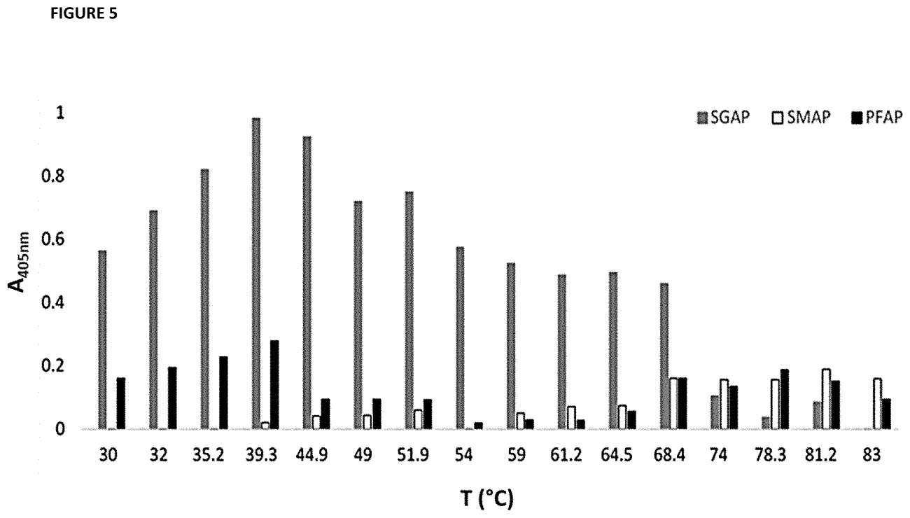

S. griseus aminopeptidase (SGAP), S. marcescens aminopeptidase (SMAP), and P. furiosus aminopeptidase (PFAP) were produced in E. coli BL21(DE3) and purified with IMAC. The activity of the purified aminopeptidases was monitored with leucine-p-nitroanilide, proline-p-nitroanilide and methionine-p-nitroanilide, respectively, at different temperatures (FIG. 5). The nitroanilide assay was performed in PBS buffer containing 1.2 mM L-leucine/L-proline/L-methionine-p-nitroaniline, 5 ng/μl aminopeptidase, 1 mM Ca²⁺ and 1 mM Zn²⁺. The results demonstrate the thermophilic or thermotolerant nature of the aminopeptidases.

### Example 6. C-Terminome Peptide Coverage Calculation Using the Leucine-, Proline-, and/or Methionine-Aminopeptidase

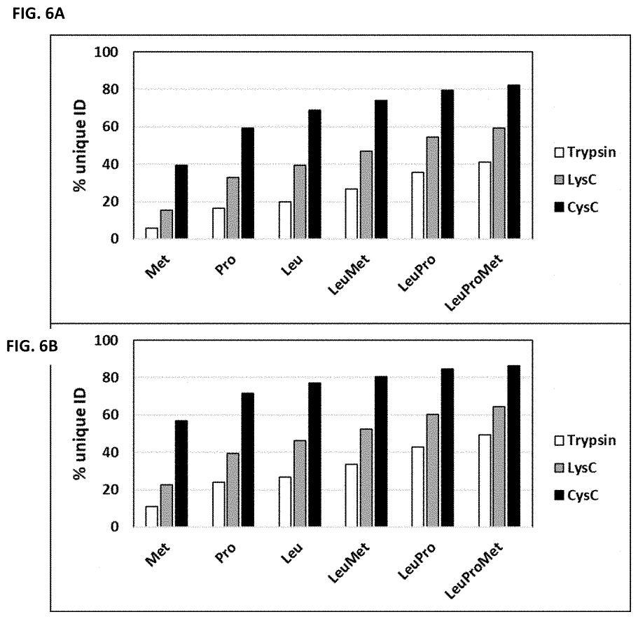

Proteins of the complete human and human plasma proteome were digested in silico with either trypsin endoprotease (R/K), lysC endoprotease (K) or CysC chemoenzymatic cysteine cleavage (C) (DeGraan-Weber and Reilly, 2018 Anal Chem 90:1608-1612). Then the C-terminal peptides were extracted and a calculation was made of the percentage of uniquely identified peptides when identifying either leucines, prolines and methionines in the sequences, or a combination thereof. When a tryptic digest is performed on the complete human proteome (20367 proteins, Uniprot (reviewed)), 42.2% of peptides are uniquely identified (FIG. 6, A). The coverage can be increased by generating longer peptides: LysC digestion leads to 59.4% unique identifications, and CysC digestion to 82.2% unique identifications (FIG. 6, A). When we performed the same calculations on the human plasma proteome (1929 proteins; Farrah et al., 2011 Mol Cell Proteomics 10:M110.006353), the coverage is slightly higher (FIG. 6, B). Here, already more than two thirds of the C-terminal peptides generated after CysC digestion can be identified, just by locating leucines in the sequences. The calculations take into account that also the length of the peptide is determined, meaning that not only the preferred amino acids (Leu, Met, Pro) are identified and cleaved, but also the cleavage of all other amino acids is achieved and detected (regardless of their identity).

1. An aminopeptidase coupled to an optical, electrical or plasmonic label for detecting the aminopeptidase, wherein the amino peptidase is selected from the group consisting of Streptomyces griseus aminopeptidase, Serratia marcescens aminopeptidase, Pyrococcus furiosus aminopeptidase, Lactobacillus helveticus X-prolyl dipeptidyl aminopeptidase, and Streptomyces griseus X-prolyl dipeptidyl aminopeptidase.

2. The aminopeptidase of claim 1, wherein the aminopeptidase is catalytically active and comprises an amino acid sequence that is at least 90% identical to and over the full length of SEQ ID No. 1, SEQ ID No. 3, SEQ ID No. 4, SEQ ID No. 5, or SEQ ID No. 6.

3. (canceled)

4. A method of identifying or categorizing the N-terminal amino acid of a polypeptide immobilized on a surface via its C-terminus, the method comprising:
a) contacting the surface immobilized polypeptide with at least one aminopeptidase which binds the N-terminal amino acid from the polypeptide;
b) measuring the residence time of the aminopeptidase on the N-terminal amino acid;
c) optionally allowing the aminopeptidase to cleave off the N-terminal amino acid;
wherein the measured residence time may be compared to a set of reference residence time values characteristic for the aminopeptidase and a set of N-terminal amino acids, so as to identify or categorize the N-terminal amino acid,
wherein the aminopeptidase is selected from the group consisting of Streptomyces griseus aminopeptidase, Serratia marcescens aminopeptidase, Pyrococcus furiosus aminopeptidase, Lactobacillus helveticus X-prolyl dipeptidyl aminopeptidase, and Streptomyces griseus X-prolyl dipeptidyl aminopeptidase.

5. The method according to claim 4, wherein the aminopeptidase is catalytically active and comprises an amino acid sequence that is at least 90% identical to and over the full length of SEQ ID No. 1, SEQ ID No. 3, SEQ ID No. 4, SEQ ID No. 5, or SEQ ID No. 6.

6. The method according to claim 4, wherein steps a) through c) or steps b) through c) are repeated one or more times.

7. The method according to claim 4, wherein the residence time is measured optically, electrically, or plasmonically and wherein the aminopeptidase is coupled to an optical, electrical or plasmonic label for detecting the aminopeptidase.

8. The method of according to claim 4, wherein the residence time of the aminopeptidase is measured for every binding event of the aminopeptidase to the N-terminal amino acid.

9. The method according to claim 11, the method further comprising determining the cleavage of the N-terminal amino acid by measuring an optical, electrical, or plasmonical signal of the surface-immobilized polypeptide, wherein a difference in optical, electrical, or plasmonical signal is indicative of cleavage of the N-terminal amino acid.

10. The method according to claim 4, the method further comprising denaturing the polypeptide prior to contacting with the aminopeptidase.

11. The method according to claim 4, wherein the polypeptide is immobilized on an active sensing surface.

12. The method of claim 11, wherein the active sensing surface is a gold surface or an amide-, carboxyl-, thiol- or azide-functionalized surface on which the polypeptide is chemically coupled.

13. A kit comprising:
a surface for immobilization of peptides and
an aminopeptidase selected from the group consisting of Streptomyces griseus aminopeptidase, Serratia marcescens aminopeptidase, and Pyrococcus furiosus aminopeptidase.

14. The kit of claim 13, wherein the aminopeptidase is catalytically active and comprises an amino acid sequence that is at least 90% identical to and over the full length of SEQ ID No. 1, SEQ ID No. 3, or SEQ ID No. 4.

15. The kit of claim 13, further comprising a X-prolyl dipeptidyl aminopeptidase.

16. The method of claim 4, wherein denaturing conditions are present during one or more of the method step.
